(19) United States
(12) Patent Application Publication
(10) Pub. No.: US 2015/0223783 A1
(43) Pub. Date: Aug. 13, 2015

(54) FEMALE URINATION COLLECTION DEVICE

(71) Applicants: Lori Eschete, Lafitte, LA (US); Marty Eschete, Lafitte, LA (US)

(72) Inventors: Lori Eschete, Lafitte, LA (US); Marty Eschete, Lafitte, LA (US)

(21) Appl. No.: 14/175,010

(22) Filed: Feb. 7, 2014

(51) Int. Cl. A61B 10/00; A61F 5/455

(52) U.S. Cl. CPC A61B 10/007; A61F 5/455

(57) ABSTRACT

A urine collection device for use by a female user when collecting a urine sample comprising a conical body, where the conical body is shaped as a funnel; a rim at a top of the conical body; a tubular base at a bottom of the conical body, where the base includes an opening to guide a urine stream from the conical body into a collection container; and a handle extending from the rim of the conical body. The urine collection device includes a central groove extending from the rim into the tubular body to guide the urine stream easily into the collection container.

### BACKGROUND OF THE INVENTION

1. Field of the Invention

The present invention relates to a device to assists a female user when providing a urine sample by funneling the urine stream into a collection container.

2. Description of Related Art

Urinalysis is a scientific procedure to test for different chemicals in a person's system; most commonly used for either medical diagnosis or drug presence. The urine analysis may be performed by applying a test strip directly into the person's urine stream, as is typically performed during a pregnancy test. Alternatively, a urine sample is collected and then the test strip is submerged or the sample is examined under the microscope. The test strips detect a specific chemical or molecule in the urine, where if the targeted chemical is present the test strip will change color. Because of the simplicity and effectiveness, urinalysis is a trusted first step in diagnosis for a variety of medical conditions.

By virtue of physiology, men have a much easier time than women in providing a urine sample. Men are able to aim their penis and go to collect the sample. Women on the other hand, have a difficult time because they do not have the means to aim, thereby approximating from where their urine stream originates. First, the women must maintain a squat pose while they position the collection cup where they think the urine stream start. Adjustments are made accordingly as they pee. Alternatively, they may start to urinate and then quickly position the cup into the urine stream. Either way they often get urine on their hands, the toilet seat, floor or clothes causing a mess that must be cleaned after collection. Another burden is when the women's arms are short or she is pregnant making the reach to get urine into the collection cup an awkward and uncomfortable endeavor.

Therefore it would be beneficial in the art to provide a means to assist a female when collecting a urine sample. It would also be desirable in the art to provide an easily disposable collection means so that after a single use the object is discarded without creating excess waste.

### SUMMARY OF THE INVENTION

In view of the foregoing disadvantages inherent in the prior art, the general purpose of the present invention is to provide a urine collection device for a female, configured to include all of the advantages of the prior art, and to overcome the drawbacks inherent therein.

Accordingly, an object of the present invention is to provide a urine collection device that includes a conical body to assists a female user to direct their urine steam into a collection container.

Another object of the present invention is to provide a female urine collection device that is disposable.

To achieve the above objects, in an aspect of the present invention, a female urine collection device is described comprising a conical body, where the conical body is shaped as a funnel; a rim at a top of the conical body; a tubular base at a bottom of the conical body, where the base includes an opening to guide a urine stream from the conical body into a collection container; and a handle extending from the rim of the conical body. The urine collection device includes a central groove extending from the rim into the tubular body to guide the urine stream easily into the collection container.

These together with other aspects of the present invention, along with the various features of novelty that characterize the present invention, are pointed out with particularity in the claims annexed hereto and form a part of this present invention. For a better understanding of the present invention, its operating advantages, and the specific objects attained by its uses, reference should be made to the accompanying drawings and descriptive matter in which there are illustrated exemplary embodiments of the present invention.

### BRIEF DESCRIPTION OF THE DRAWINGS

The advantages and features of the present invention will become better understood with reference to the following detailed description and claims taken in conjunction with the accompanying drawings, wherein like elements are identified with like symbols, and in which:

FIG. 1 depicts a side view of a female urination collection device in accordance with an exemplary embodiment of the present invention;

FIG. 2 depicts a perspective view of a female urination collection device in accordance with an exemplary embodiment of the present invention; and

FIG. 3 depicts a top view of a female urination collection device in accordance with an exemplary embodiment of the present invention.

Like reference numerals refer to like parts throughout the description of several views of the drawings.

### DETAILED DESCRIPTION OF THE DRAWINGS

The present invention relates to a device to assists a female user when providing a urine sample by funneling the urine stream into a collection container. The present invention provides a female urination collection device to simplify urine sampling by aiding the women to urinate into a collection container without creating a mess. The female urination collection device includes a conical funnel of a complimentary shape to the female genitalia. The funnel rests comfortably against the user's body and guides the urine stream into a collection container. With the female urination collection device the women has a sanitary alternative to standard methods of urination collection. They can rest assure that their hands will remain dry and that they will not spill while providing a urine sample.

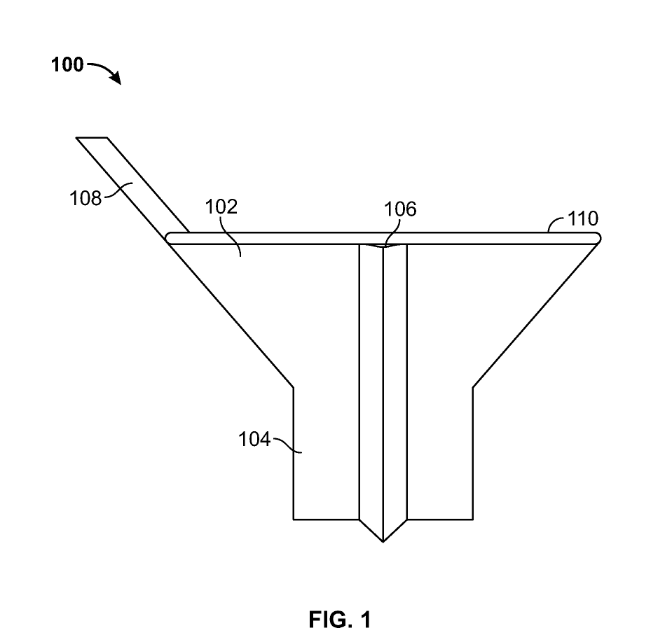

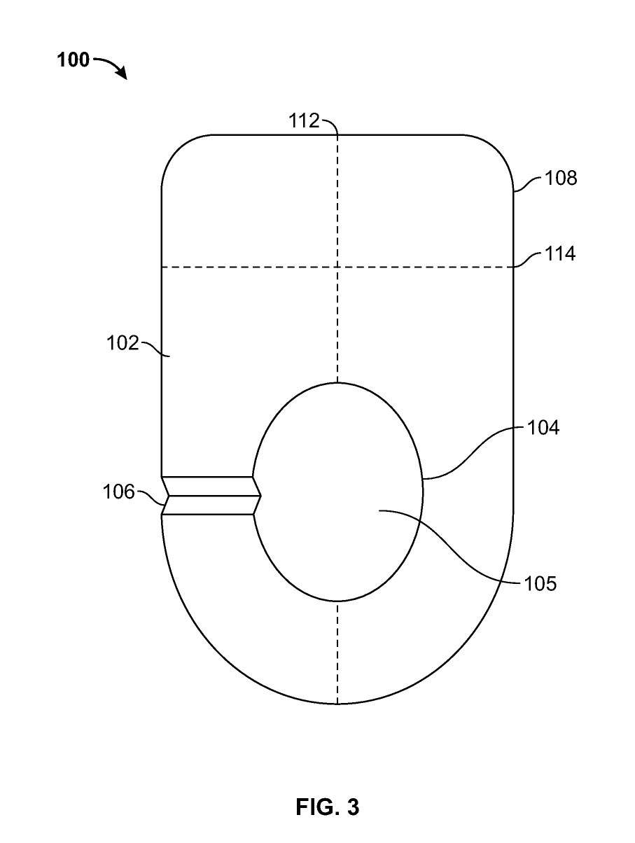

Turning now descriptively to the drawings, referring to FIG. 1, a side view of a female urination collection device 100 is shown in accordance with an exemplary embodiment of the present invention. The female urination collection device 100 includes a conical body 102 connected to a tubular base 104. The conical body 102 may comprise an oval shape (as illustrated in FIG. 3) for a complimentary shape against a female user's body. This way a rim 110 of the conical body 102 rests against the user's body. The rim 110 may be rounded to for a comfortable feel against the skin, and to prevent any sharp edges from harming the user. With a comfortable and snug fit the urine stream does not splash or dribbling onto the user's fingers or outside of the collection container. The conical body 102 may be made from a recyclable plastic, recyclable rubber, waxed lined paper or another recyclable or biodegradable material.

A central groove 106 may be positioned within the conical body 102 extending from the rim 110 down into the base 104. The central groove 106 guides a urine stream directly into the conical body 102 and the base 104 where the urine is then deposited into an underlying collection container. The central groove 106 may be folded accordion style to create a V-shaped trough that guides the urine stream.

Extending from the rim 110 of the conical body 102 may be a handle 108. The handle 108 serves as a gripping portion for the user thereby keeping their hands away from the urine stream. Additionally, the handle 108 may shield the user's hands from any splash created while collecting a urine sample.

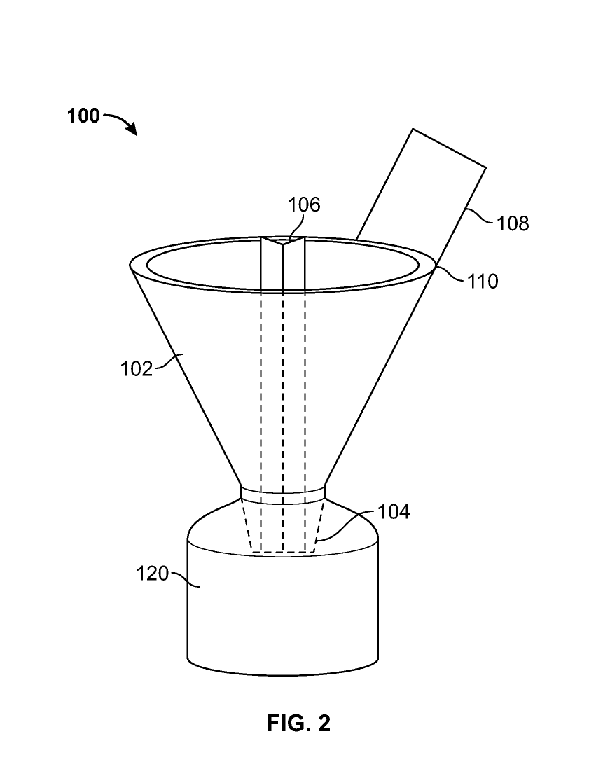

Referring now to FIG. 2, a perspective view of the female urine collection device 100 is illustrated with a collection container 120. The tubular base 104 of the conical body 102 is easily positioned within the collection container 120. This way the urine stream is guided into the collection container without risk of spilling or splashing onto the user.

Referring now to FIG. 3 a top view of the female urine collection device 100 is shown in accordance with an exemplary embodiment of the present invention. The opening 105 of the base 104 may be oval shaped to reflect the complementary shape of the conical body 102 to the female user's body. For easy storage, the female urine collection device 100 may be folded along the dotted lines 112, 114. A bifurcated fold 112 runs through the conical body 102, the base 104 and the handle 108 to completely fold the female urine collection device 100 in half. A handle fold 114 enables the handle 108 to fold against the conical body 102. With the folds 112, 114 the female urine collection device 100 is easily flattened for storage in a box containing a multitude of single-use devices 100, for example at a pharmacy, doctor's office or even at home.

With the female urine collection device the user is relieved of the stress of having to collect a pee sample. They are able to efficiently and successfully guide the urine into the collection container, without worry that their hands or clothes will be soiled. Since the conical body 102 is disposable and/or recyclable no excess waste is created making the female urine collection device 100 an ideal funneling tool.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

What is claimed is:

1. A urine collection device for use by a female user when collecting a urine sample comprising:
a. a conical body, where the conical body is shaped as a funnel;
b. a rim at a top of the conical body;
c. a tubular base at a bottom of the conical body, where the base includes an opening to guide a urine stream from the conical body into a collection container; and
d. a handle extending from the rim of the conical body.

2. The urine collection device according to claim 1, where the conical body includes a central groove, where the central groove extends from the rim to the opening of the tubular base.

3. The urine collection device according to claim 2, where the central groove includes an accordion fold to guide the urine stream.

4. The urine collection device according to claim 1, where the conical body is disposable.

5. The urine collection device according to claim 4, where the conical body is made from a recyclable material.

6. The urine collection device according to claim 1, where the rim and the conical body are oval shaped for a complementary shape against the female user's body.

7. The urine collection device according to claim 1, where the handle serves as a shield for the female user's hand.

8. The urine collection device according to claim 1, where the urine collection device includes a bifurcating fold extending through the handle, the conical body and the tubular base to fold the urine collection device in half.

9. The urine collection device according to claim 9, where a plurality of folded urine collection devices are provided in a storage box.

10. The urine collection device according to claim 1, where the handle folds along a handle fold.

11. The urine collection device according to claim 1, where the rim is rounded.
